(19) United States
(12) Patent Application Publication
(10) Pub. No.: US 2008/0125718 A1
(43) Pub. Date: May 29, 2008

(54) Tubular Member Fixing Device

(75) Inventors: Hiroaki Tsuchiya, Fukuroi-shi (JP); Hiroyuki Onagi, Fukuroi-shi (JP)

Correspondence Address: TYCO HEALTHCARE - EDWARD S. JARMOLOWICZ, 15 HAMPSHIRE STREET, MANSFIELD, MA, 02048, US

(73) Assignee: Nippon Sherwood Medical Industries Ltd., Shizuoka (JP)

(21) Appl. No.: 11/568,397

(22) PCT Filed: Apr. 26, 2005
(86) PCT No.: PCT/EP05/04465

§ 371 Date: Aug. 23, 2007

(30) Foreign Application Priority Data
Apr. 28, 2004 (JP) 2004-133308

(51) Int. Cl. A61M 5/32

(52) U.S. Cl. 604/174

(57) ABSTRACT

[Problem] To provide a fixing device for a tubular member such as a catheter which is easy to operate, has parts which are easy to tighten, which makes it easy to recognize whether the parts are securely fixed, which is small in size, and which causes minimum irritation of the skin.

[Means for Resolution] A tubular member fixing device includes: a second fixing portion (20) being formed of soft material and including a retaining portion (22) capable of opening and closing for clamping the tubular member (30) and a second base member (21) formed integrally with the retaining portion (22) at a fitting recess (24); and a first fixing portion (1) being formed of material harder than that of the second fixing portion (20) and including a first base member (2) having a covering portion (3) for covering the outer surface (22d) of the retaining portion (22) so as to be capable of opening and closing the opening-closing side of the retaining portion (22) of the second fixing portion (20), and a fitting joint (2a) formed integrally with the covering portion (3) for fitting into the fitting recess (24) of the second fixing portion (20), and the second fixing portion (20) and the first fixing portion (1) are fitted to abut against each other.

### TECHNICAL FIELD

The present invention relates to a tubular member fixing device for preventing accidental fore-and-aft movement of a tubular member such as a catheter that is inserted and indwelled in a body for transfusion injecting, body fluid circulating or draining, and fixing the tubular member on the body surface.

### BACKGROUND ART

In recent years, treatment or diagnosis using a tubular member such as a catheter or a tube have been widely employed. Such a tubular member is hollow, and is used for injecting drug solution into a body or draining out of the body. Examples of the tubular member include, for example, a gastric tube catheter, bronchial tube, middle cardiac vein catheter, and suction catheter.

FIG. 15 is a perspective view showing an example of a tubular member fixing device for fixing a tubular member such as the catheter in a related art. A fixing device 40 for fixing the tubular member such as the catheter can fix the tubular member by inserting the tubular member via a slit 42 into a cylindrical retaining member 41 for retaining the tubular member and then fixing a first wing portion 43 and a second wing portion 44 in an overlapped state, so that the slit 42 is clogged and the retaining member 41 is reduced in diameter by a contracting force exerted on the exterior thereof.

Such a device is disclosed in the document JP-A-2002-360705.

The configuration of the prior art was problematic in that, during re-adjustments of the catheter after it was attached to the skin, a high concentration of force was applied on one side to open the device, which, through cantilever effect, irritated the sature point apposite the closure side.

### DISCLOSURE OF THE INVENTION

### Problems that the Invention is to Solve

In the tubular member fixing device in the related art, there are problems in that it is difficult to tighten the tubular member such as the catheter, and to recognize whether it is securely tightened or not by the feel. Furthermore, the size of the catheter fixing device is large, and the fixed portion irritates the skin significantly.

In order to solve the above described problems, it is an object of the present invention to provide a tubular member fixing device which is easy to operate, easy to tighten, and easy to recognize whether it is securely fixed or not, which can be small in size, and in which skin irritation is minimized.

### Means for Solving the Problems

A tubular member fixing device according to the present invention includes: a first fixing portion, a second fixing portion, first and second base members, and first and second retaining portions. The second fixing portion is formed of soft material and includes a second retaining portion for clamping the tubular member so as to be capable of opening and closing, as well as a second base member formed integrally with the retaining portion via a fitting recess. The first fixing portion is formed of material harder than that of the second fixing portion and includes a first base member having a covering portion for covering the outer surface of the retaining portion. The covering portion of the first fixing member is in abutment with the opening-closing side of the retaining portion of the second fixing portion so that the retaining portion is capable of opening and closing. A fitting joint is formed integrally with the covering portion of the first fixing member for fitting into the fitting recess of the second fixing portion, and the second fixing portion and the first fixing portion are fitted to each other so as to abut against each other.

Furthermore, the second fixing portion including the retaining portion is cylindrical in the closed state, and the second base member is of planar shape and comprises a fitting stopper. The first fixing portion, including a covering portion which opens and closes with a hinge portion, has a semi-cylindrical surface for covering the outer surface of the retaining portion. A fitting joint having a semi-cylindrical surface is to be joined with the covering portion via the hinge portion. The first base members of a planar shape comes into abutment on both sides thereof with the second base member.

The tubular member fixing device further includes a first engaging portion provided at a distal end of the covering portion, and a second engaging portion provided on the side toward the fitting joint, and is configured so that the engaging portions engage with each other.

The tubular member fixing device according to the present invention may also be configured in such a manner that a pair of covering portions are disposed to face each other, and the pair of covering portions include first and second engaging portions so as to be engaged with each other. Furthermore, the first and second engaging portions are engaged on the side of the top surface.

The tubular member fixing device may also include a plurality of projections provided on the outer surface of the retaining portion so that the projections and the covering portion come into abutment with each other.

The material of the first fixing portion may be polypropylene, polyethylene, or another material with similar properties.

The material of the second fixing portion may be soft vinyl chloride, rubber, or another material with similar properties.

The tubular member fixing device according to the present invention may be configured in such a manner that the tubular member is a catheter.

### Advantage of the Invention

The tubular member fixing device according to the present invention has advantages such as that it can be recognized easily by the feel that the tubular member such as the catheter is fixed, irritation of the skin is minimized, and the fixing position can be corrected easily. In particular, the two parts which are formed of different materials can be fitted in advance into a single unit, whereby the complex operation of manipulating two different parts when in use may be avoided. With this configuration, the fixing operation of the tubular member can be achieved easily without dropping one of the parts during operation.

Through a construction in which the device of the present invention is made of two distinct components or portions of a single component—a softer portion which is in contact with the skin and grips the tubular member, and a harder portion providing stability—the present invention reduces skin irritation and increases reliability simultaneously. In keeping with the scope of this disclosure, one can envision other constructions in which a single relatively soft polymeric molding is provided with locally hardened engagement members on respective covering portions which can be latched together by squeezing between thumb and finger, and unlatched, without putting pressure on the base plate.

### BEST MODE FOR CARRYING OUT THE INVENTION

### First Embodiment

FIG. 1 is a front view of the first embodiment of the present invention, a catheter fixing device with an outer fixing member and an inner fixing member fitted and integrated with each other. FIG. 2 is a plan view of FIG. 1. FIG. 3 is a side view of FIG. 1 when viewed in the direction A. FIG. 4 is a front view of the outer fixing member which constitutes the catheter fixing device in FIG. 1. FIG. 5 is a plan view of FIG. 4. FIG. 6 is a side view of FIG. 4 when viewed in the direction B. FIG. 7 is a front view of the inner fixing member which constitutes the catheter fixing device in FIG. 1. FIG. 8 is a plan view of FIG. 7, and FIG. 9 is a side view of FIG. 7 in the direction C.

The catheter fixing device includes an outer fixing member 1 (FIG. 4 to FIG. 6) formed of material which is relatively hard and is hard to deform so as not to be broken even when it is bent, and an inner fixing member 20 (FIG. 7 to FIG. 9) formed of flexible soft vinyl chloride, and is formed by combining and fitting these parts into a single unit (FIG. 1 to FIG. 3).

The outer fixing member 1 which constitutes the first part of the catheter fixing device includes a first base member 2 and a covering portion 3 formed integrally with the first base member 2, as shown in FIG. 4 to FIG. 6. The first base member 2 includes a fitting joint 2a which connects with the covering member 3, and flat portions 2b, 2c of a planar shape located on both sides of the fitting joint 2a which project therefrom in a substantially triangular shape. At the bottom surface 2d of both flat portions 2b, 2c are formed engaging protrusions 4a, 4b of substantially columnar shape, and both flat portions 2b, 2c and the engaging protrusions 4a, 4b are formed with holes 5a, 5b which penetrate through flat portions 2b, 2c and the engaging protrusions 4a, 4b.

The fitting joint 2a of the first base member 2 includes a fitting recess 6 having a substantially semi-cylindrical surface, and the covering portion 3 having a substantially semi-cylindrical surface is integrally connected to the other end of the fitting recess 6 via a hinge portion 7. The covering portion 3 is formed with a claw-shaped first engaging portion 9 at the distal end thereof, which engages with a claw-shaped second engaging portion 10 formed at the end of the fitting recess 6 to define a substantially cylindrical hollow 11 in the engaged state.

As shown in FIG. 7 to FIG. 9, the inner fixing member 20 constituting the second part of the catheter fixing device includes a planar shaped second base member 21 and a retaining portion 22 of the catheter 30 formed integrally with the second base member 21 via a fitting recess 24 which is provided with a fitting stopper 23. The second base member 21 includes a center planer portion 21a provided with the fitting stopper 23 which communicates with the retaining portion 22, and both-side flat portions 21b, 21c, which are located on both sides thereof and which project therefrom in a generally triangular shape. The both-side flat portions 21b, 21c are provided with circular engaging holes 25a, 25b which fit on the engaging projections 4a, 4b of the first fixing portion 1. The retaining portion 22 has substantially inverted C-shape in cross-section in the opened state, and is configured so that abutting portions 22a, 22b come into abutment to define substantially a cylindrical shape, in which the catheter 30 is clamped in a hollow 26 when closed. The retaining portion 22 may be one which opens into the inverted C-shape from the closed state when force is applied.

As shown in FIG. 1 to FIG. 3, in the catheter fixing device in a state in which the inner fixing member 20 and the outer fixing member 1 are fitted to each other, an upper surface 21d of the second base member 21 of the inner fixing member 20 abuts against the bottom surface 2d of the first base member 2 of the outer fixing member 1, and the engaging protrusions 4a, 4b of the outer fixing member 1 are fitted into the engaging holes 25a, 25b of the inner fixing member 20. An outer surface 22c of the retaining portion 22 of the inner fixing member 20 toward the fitting recess 24 comes into abutment with an inner surface 6a of a fitting recess 6 of the fitting joint 2a of the outer fixing member 1. In addition, the outer surface 22d which is located on the side of the abutting portions 22a, 22b of the retaining portion 22 of the inner fixing member 20 is adapted to abut against the inner surface 3a of covering portion 3 when the covering portion 3 of the outer fixing member 1 is closed.

In the description above, the material of the outer fixing member 1 has been designated as polypropylene. However, it may be formed of plastic resin such as polyethylene, ABS, polycarbonate, hard vinyl chloride, or polystyrene, or may be formed of metal. The material of the inner fixing member 20 has been designated as soft vinyl chloride; however, it may be formed of rubber such as silicone rubber, elastomer, or EVA.

The operation of the catheter fixing device configured as described above will be described. First, a method of assembling the catheter fixing device will be described. The inner fixing member 20 in the opened state shown in FIG. 7 is inserted from the back side of the drawing of the outer fixing member 1 in the opened state shown in FIG. 4 and fitted thereto. In other words, the inner fixing member 20 is slid and inserted into the outer fixing member 1 so that the fitting recess 24 of the inner fixing member 20 clamps the fitting joint 2a of the outer fixing member 1. In this assembled position, the upper surface 21d of the second base member 21 is abutted with the lower surface 2d of the first base member 2 of the outer fixing member 1, and the outer surface 22c of the inner fixing member 20 is abutted with the inner surface 6a of the fitting recess 6 of the outer fixing member 1. Accordingly, the inner fixing member 20 is inserted into the outer fixing member 1 only up to a certain distance, and the fitting stopper 23 of the inner fixing member 20 comes into abutment with the side surface of the fitting joint 2a and hence is stopped, and, as shown in FIG. 1 to FIG. 3, the inner fixing member 20 is fitted and integrated with the outer fixing member 1.

A method of using the catheter fixing device will now be described. As shown in FIG. 1, when the catheter 30 is placed in the hollow 26 of the retaining portion 22 of the inner fixing member 20 in the opened state, and then the cover portion 3 of the outer fixing member 1 is closed, the claw-shaped first and second engaging portions 9, 10 engage and the catheter fixing device comes into the closed state. At this time, as shown in FIG. 10, the retaining portion 22 of the inner fixing member 20 is pressed by the covering portion 3 of the outer fixing member 1, and hence the abutting portions 22a, 22b of the retaining portion 22 abut against each other, whereby the retaining portion 22 is tightened from the outside and deformed into substantially a cylindrical shape, so that the catheter 30 is clamped and fixed.

Subsequently, a suture thread is passed through the holes 5a, 5b, and the first and second base members 2, 21 of the catheter fixing device are fixed to the skin, and in this state, liquid is flowed through the catheter 30.

When a force is exerted in the direction in which the covering portion 3 bends, fitting of the outer fixing member 1 can be released. In other words, the device may be opened by pressing in approximately the center of the device, in a direction normal to the skin. In this manner, the force is dispersed evenly throughout the device, leading to a more comfortable tubular member 30 exchange and less skin irritation for the patient.

According to the first embodiment, since the catheter fixing device is formed by fitting parts formed of different materials, the catheter can be fixed, and the fixing operation of the catheter can be performed easily. In other words, since the two parts of different materials are fitted and integrated in advance, complex operations such as manipulation of two different parts when using may be avoided, so that the fixing operation of the catheter can be achieved easily and reliably without dropping one of the parts during operation. Also, since the claw-shaped first and second engaging portions 9, 10 of the outer fixing member 1 are engaged and fixed, the fact that the catheter is fixed can be recognized easily by the feel. In addition, since the lower surface of the catheter fixing device is formed by the inner fixing member 20 of soft material, skin irritation by the catheter fixing portion is minimized. Also, by releasing the fixation of the first and second engaging portions 9, 10, the fixing position can be corrected easily.

### Second Embodiment

FIG. 12 is a front view of the outer fixing member 1 constituting the catheter fixing device according to a second embodiment. In the first embodiment, the first engaging portion 9 is provided on the covering portion 3 of the outer fixing member 1, and the second engaging portion 10 is further provided on the side of the fitting joint 2a, so that the first and second engaging portions 9, 10 are engaged at a side surface of the catheter fixing device. However, in the second embodiment, a pair of covering portions 3a, 3b are provided so as to face each other, and these covering portions 3a, 3b are engaged at the top position of the catheter fixing portion. When the device is attached to the skin, and the tubular member 30 requires adjustment, the device can be opened by squeezing the covering portions 3a, 3b toward one another. In this manner, no force normal to the skin is applied, thus achieving a pain-free, irritation-free catheter 30 exchange for the patient.

In FIG. 12, the outer fixing member 1 is provided with a pair of covering portions 3a, 3b of substantially an arcuate shape connected integrally to each other via hinge portions 7a, 7b on both sides of the fitting joint 2a of the first base member 2. Formed at the distal ends of the respective covers 3a, 3b are the first and second engaging portions 9, 10, and the first and second engaging portions 9, 10 engage at a position near the top surface as shown in FIG. 13, so as to define a substantially column-shaped hollow 11.

In this case, the position of the abutting portions 22a, 22b of the retaining portion 22 of the inner fixing member 20 is also at a position near the top surface corresponding to the position of the first and second engaging portions 9, 10 of the outer fixing member 1. However, the invention is not limited thereto, and it may be a position near the side surface of the retaining portion 22, or may be other position as long as the catheter 30 can be placed.

Other structures, operation, and effects of the second embodiment are substantially the same as described in the first embodiment, and hence descriptions of these features will be omitted.

### Third Embodiment

FIG. 14 is a front view of the inner fixing member 20 according to a third embodiment of the present invention. In the first embodiment, the retaining portion 22 is substantially cylindrical, having constant thickness in the closed state. However, in the third embodiment, protrusions 22e oriented in the axial direction are provided at regular intervals on the outer surface of the substantially cylindrical retaining portion 22. In this case, the thickness of the retaining portion 22 other than the projections 22e is configured to be thinner than the thickness of the retaining portion 22 shown in the first embodiment.

In the arrangement as described above, a pressing energy exerted by the covering portion 3 when the outer surface of the retaining portion 22 is covered by the covering portion 3 of the outer fixing member 1 can be absorbed. In particular, when the tubular member 30 is thick, the pressure generated thereby can be absorbed by the projections 22e of the retaining portion 22. In this embodiment, it is to be appreciated that the force required to close the covering portion is reduced, without sacrificing a reliable hold of the catheter.

Other structures, operation and effects of the third embodiment are substantially the same as the case shown in the first or second embodiment, descriptions thereof will be omitted.

### BRIEF DESCRIPTION OF THE DRAWINGS

FIG. 1 is a front view of a catheter fixing device according to a first embodiment of the present invention.

FIG. 2 is a plan view of FIG. 1

FIG. 3 is a side view of FIG. 1 when viewed in the direction A.

FIG. 4 is a front view of an outer fixing member which constitutes the catheter fixing device in FIG. 1.

FIG. 5 is a plan view of FIG. 4.

FIG. 6 is a side view of FIG. 4 when viewed in the direction B.

FIG. 7 is a front view of an inner fixing member which constitutes the catheter fixing device in FIG. 1.

FIG. 8 is a plan view of FIG. 5.

FIG. 9 is a side view of FIG. 7 when viewed in the direction C.

FIG. 10 is a drawing explaining the operation of a device in FIG. 1.

FIG. 11 is an explanatory drawing of a principal portion of FIG. 10.

FIG. 12 is a front view of the outer fixing member constituting the catheter fixing device according to the second embodiment of the present invention.

FIG. 13 is an explanatory drawing showing a principal portion of FIG. 12.

FIG. 14 is a front view of the inner fixing member constituting the catheter fixing device according to the third embodiment of the present invention.

FIG. 15 is a perspective view showing an example of the catheter fixing device in the related art.

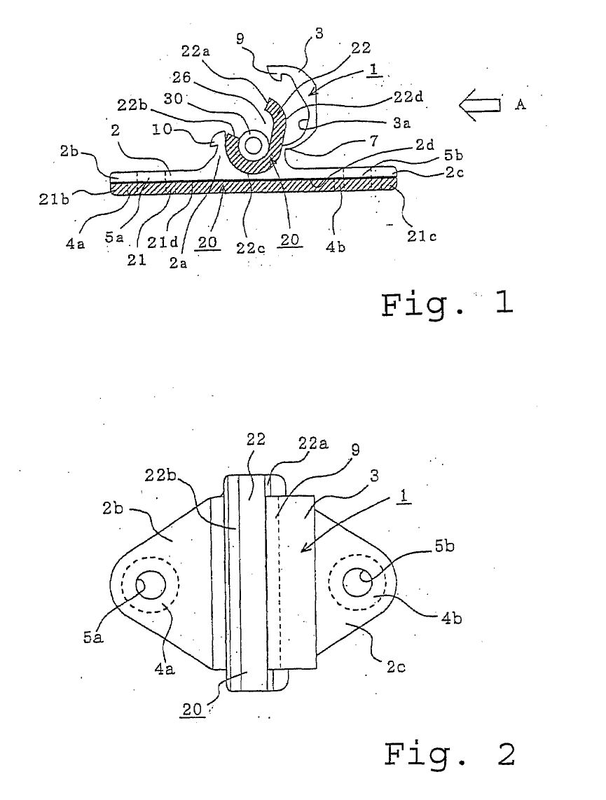

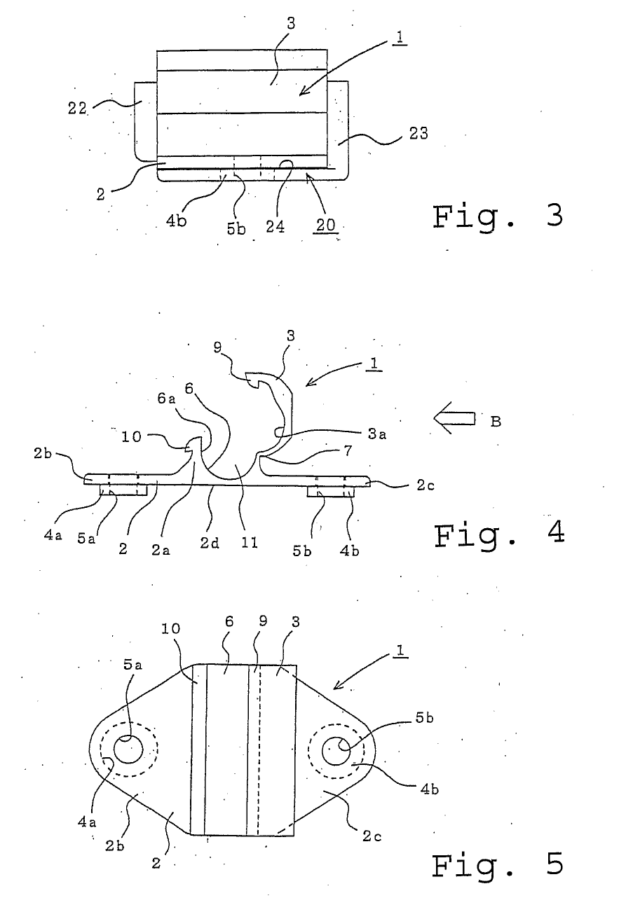

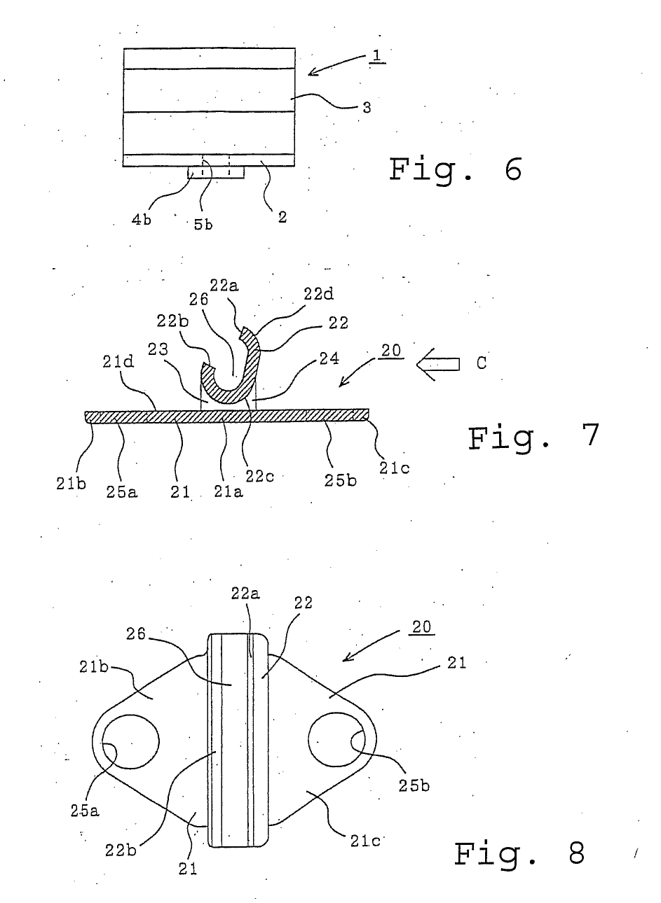

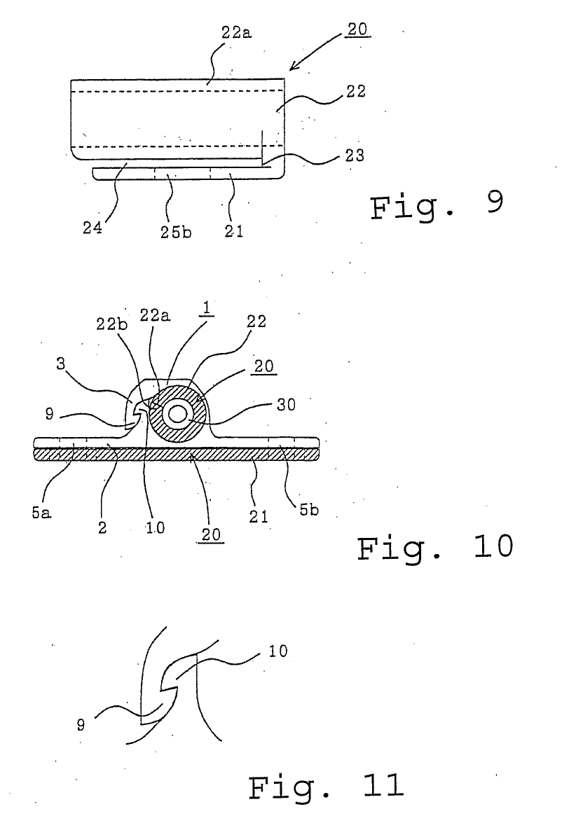

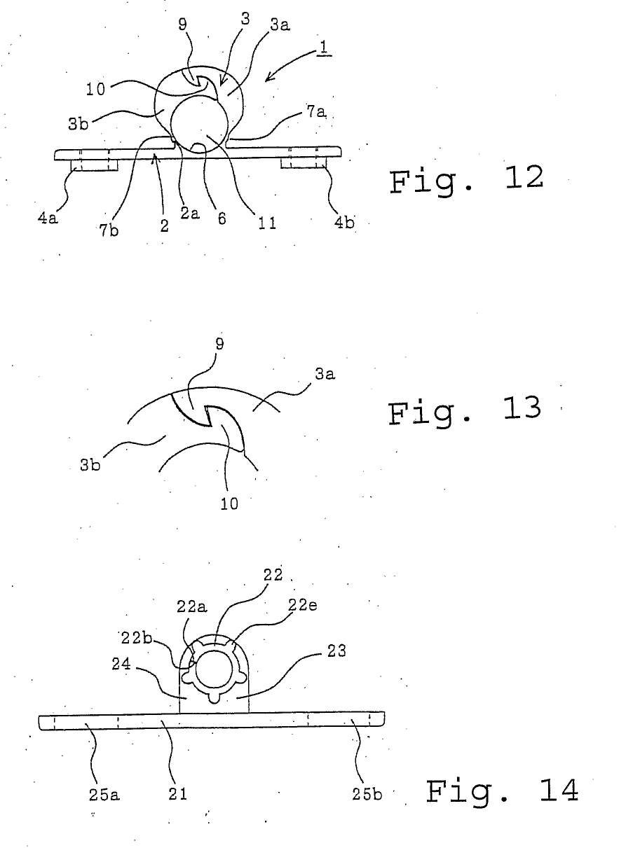

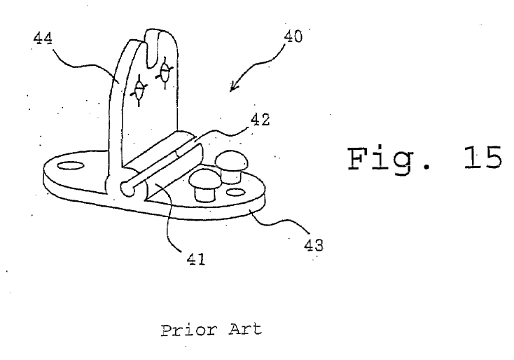

### DESCRIPTION OF THE REFERENCE NUMERALS AND SIGNS

1 outer fixing member (first fixing portion), 2 first base member, 2a fitting joint, 2b, 2c, 21b, 21c flat portion of base member, 3, 3a, 3b covering portion, 7, 7a, 7b hinge portion, 9 first engaging portion, 10 second engaging portion, 20 inner fixing member (second fixing portion), 21 second base member, 22 retaining portion, 22c, 22d outer surface of retaining portion, 22e projections, 23 fitting stopper, 24 fitting recess, 30 catheter (tubular member)

1. A tubular member fixing device comprising:
a first fixing portion and a second fixing portion,
the second fixing portion being formed of soft material and including a retaining portion for clamping the tubular member so as to be capable of opening and closing and a second base member formed integrally with the retaining portion; and
the first fixing portion being formed of material harder than that of the second fixing portion and including a first base member and having a covering portion for covering the outer surface of a retaining portion, which is in abutment with the opening-closing side of the retaining portion of the second fixing portion so that the retaining portion of the second fixing portion is capable of opening and closing, and a fitting joint formed integrally with the covering portion;
characterized in that the second fixing portion and the first fixing portion are fitted to each other so as to abut against each other.

2. The tubular member fixing device of claim 1, wherein the base member is formed integrally with the retaining portion via a fitting recess.

3. The tubular member fixing device of claim 2, wherein fitting joint of the first fixing portion fits into the fitting recess of the second fixing portion.

4. The tubular member fixing device according to claim 3 comprising:
the second fixing portion including the retaining portion in a cylindrical shape in the closed state and a second base member of planar shape formed integrally with the retaining portion via the fitting recess equipped with a fitting stopper; and
the first fixing portion including a covering portion which opens and closes with a hinge portion and has a semi-cylindrical surface for covering the outer surface of the retaining portion, a fitting joint having a semi-cylindrical surface to be joined with the covering portion via the hinge portion, and the first base members of a planar shape which come into abutment on both sides thereof with the second base member.

5. The tubular member fixing device according to claim 1, comprising a first engaging portion provided at a distal end of the covering portion, and a second engaging portion provided on the side toward the fitting joint, characterized in that the engaging portions are engaged with each other.

6. The tubular member fixing device according to claim 1, characterized in that a pair of covering portions are disposed facing each other, and the pair of covering portions comprises first and second engaging portions at the respective distal ends thereof so as to be engaged with each other.

7. The tubular member fixing device according to claim 6, wherein the first and second engaging portions are engaged on the top surface.

8. The tubular member fixing device according to claim 1 comprising a plurality of projections provided on the outer surface of the retaining portion so that the projections and the covering portion come into abutment with each other.

9. A method of using a catheter fixing device comprising the steps of:
placing the catheter in the holding portion of the device in the opened state, and
squeezing the ends of the covering portion of the device together, thus locking the ends together.

10. The method of claim 9 further comprising the steps of squeezing the ends of the covering portion, which are already in the closed state, thus unlatching the ends and opening the device.

11. A fixing device for a catheter, which comprises opposed catheter covering portions upstanding from a base plate, to be fixed to a skin surface, the respective covering portions terminating in co-operating latching portions, the latching portions being formed of a material harder than that of the covering portions.

12. The fixing device of claim 11 wherein the portion of the base plate abutting the skin is formed of a material which is softer than that of the latching portions.

13. The fixing device of claim 11, wherein the portion abutting the catheter is formed of a material which is softer than that of the latching portions.

14. The fixing device of claim 11, wherein the latching portions are engaged by squeezing said portions between thumb and finger.
